(19) United States
(12) Patent Application Publication
(10) Pub. No.: US 2003/0068829 A1
(43) Pub. Date: Apr. 10, 2003

(54) High throughput crystallographic screening of materials

(75) Inventors: Daniel M. Giaquinta, Mountain View, CA (US); Eric D. Carlson, Cupertino, CA (US)

Correspondence Address: Eric M. Dobrusin, Dobrusin & Thennisch PC, Suite 311, 401 South Old Woodward Avenue, Birmingham, MI, 48009, US

(73) Assignee: Symyx Technologies, Inc.

(21) Appl. No.: 10/177,185

(22) Filed: Jun. 21, 2002

Related U.S. Application Data
(60) Provisional application No. 60/300,792, filed on Jun. 25, 2001.

(51) Int. Cl. G01N 33/00

(52) U.S. Cl. 436/173

(57) ABSTRACT

A method for characterization of a solid material sample, including the steps of providing a liquid medium for delivering a solid material sample to a porous substrate. Solid sample is separated from the liquid medium by filtering through the porous substrate. Analysis such as x-ray analysis of the solid sample is conducted while the sample resides on the porous substrate.

### CLAIM OF BENEFIT OF FILING DATE

The present application claims the benefit of the filing date of U.S. Provisional Application Serial No. 60/300,792, filed Jun. 25, 2001, the contents of which are hereby incorporated by reference.

### FIELD OF THE INVENTION

The present invention generally relates to methods for high throughput screening of materials, and more particularly to the high throughput crystallographic screening of libraries of solid materials for the discovery of new materials or the rapid characterization of existing materials.

### BACKGROUND OF THE INVENTION

The discovery of new materials with novel chemical and physical properties often leads to the development of new and useful technologies. Over forty years ago, for example, the preparation of single crystal semiconductors transformed the electronics industry. Currently, there is a tremendous amount of activity being carried out in the areas of new solid materials. Unfortunately, even though the chemistry of extended solids has been extensively explored, few general principles have emerged that allow one to predict with certainty composition, structure and reaction pathways for the synthesis of such solid state compounds, compositions or structures. Moreover, it is difficult to predict a priori the physical properties or the microstructure that a particular material will possess.

Clearly, the preparation of new materials with novel chemical and physical properties is at best happenstance with our current level of understanding. Consequently, the discovery of new materials depends largely on the ability to synthesize and analyze new materials, compounds, compositions or structures. Given approximately 100 elements in the periodic table that can be used to make such compositions consisting of three, four, five, six or more elements, the universe of possible new compounds remains largely unexplored. As such, there exists a need in the art for a more efficient, economical and systematic approach for the synthesis of potential new compounds, compositions or structures (e.g., materials) and for the screening of such new materials, or even of existing materials, for structural information potentially bearing upon the useful properties of the materials.

Schultz et al., in U.S. Pat. No. 5,985,356 entitled “Combinatorial Synthesis of Novel Materials” disclose methods for preparing and screening arrays of crystalline materials for combinatorial material science applications, including screening of materials on a substrate, and is incorporated herein by reference.

Heretofore the efficient crystallographic analysis of a combinatorial library of materials, when the materials are present in solution or otherwise in a fluidic media (e.g., as a precipitate or particulate) has been generally hampered by the practice of synthesizing the individual library members (e.g., on a first substrate) and then, individually, isolating or transferring each respective library member onto a second substrate for analysis, such as by x-ray diffraction. Accordingly, prior to the present invention, there was a need for a more efficient system for the rapid synthesis, separation and crystallographic characterization of individual members of such a combinatorial library.

One x-ray diffraction screen for combinatorial chemistry is described in He et al., “XRD Rapid Screening System for Combinatorial Chemistry”, Advances in X-ray Analysis, Vol. 44, Proceedings of the 49^{th} Annual Denver X-ray Conference (Jul. 31-Aug. 4, 2000). See also, Isaacs et al, “Synchrotron X-Ray Microbeam Diagnostics of Combinatorial Synthesis”, Applied Physics Letters, Vol. 73, No. 13, pp. 1820-22 (Sep. 28, 1998).

In WO99/59716 (published Nov. 25, 1999), there purports to describe a process for the wet chemical production of a plurality of libraries of materials consisting of solids, whereby the solids are separated from reaction mixtures in so called “micro-reaction chambers” on a base plate, which also purports to serve at the same time as a substrate for the library.

This invention provides methods and apparatus for the synthesis of combinatorial libraries or arrays on or in suitable substrates by effectively utilizing a certain combination of steps or structures. The invention can be used to make known materials or new materials.

### SUMMARY OF THE INVENTION

In one aspect, this invention provides a method for crystallographically screening a plurality of solid samples, including the steps of providing a flowable medium for delivering a solid material sample to a porous substrate. Solid sample is separated from the liquid medium by filtering through the porous substrate, such that solid sample is deposited on the porous substrate. Analysis such as x-ray analysis (e.g., x-ray diffraction) or other beam radiation analysis of the solid sample is conducted while the sample resides on the porous substrate.

In another aspect, the present invention provides an apparatus including: a substrate holder having at least four apertures and being adapted to receive and support a porous substrate having a first side and a second side, the substrate holder being further adapted to provide for fluid communication between the samples and the first side of the porous substrate. A receptacle having at least one collection cavity is adapted to provide fluid communication between the collection cavity and the second side of the porous substrate. A source for providing a pressure gradient across said porous substrate is incorporated, such that the samples in fluid communication with the first sides of the porous substrates can be simultaneously filtered through the porous substrates to separate solid-phase components of the samples from liquid phases thereof, and thereby deposit the solid phase components of the samples on the first side of the porous substrate.

In the context of new materials discovery research, the methods and apparatus of the present invention will provide an efficient and rapid approach for the crystallographic analysis of solid materials. The present invention thus readily permits for the use of high throughput screens to identify potentially significant new materials or to characterize existing materials, whether the materials are crystalline, amorphous or a combination. The present invention permits for the use of liquid chemistry techniques for the rapid and efficient synthesis of small quantities of sample materials, including those prepared using automated instruments. The present invention is also useful for identifying whether or not a crystalline material is present, and without necessarily any regard for the particular crystal structure of the resulting material. The present invention is also useful for analyzing other morphological properties of solid materials. In short, the present invention provides useful analytical approaches for determining crystallization, phase transformations, and crystal structure resolution.

### DESCRIPTION OF DRAWINGS

FIG. 1 is a schematic of an illustrative research system in accordance with the present invention.

FIG. 2 is an exploded perspective view of one illustrative sample collection system of the present invention.

FIG. 3 is a perspective view of one illustrative library deposited on a porous substrate.

FIG. 4 is a side view illustrating a seal with a porous substrate.

### DETAILED DESCRIPTION OF PREFERRED EMBODIMENT

The following terms are intended to have the following general meanings as they are used herein:

Region: In the context of the present invention, a region is a localized area on a porous substrate intended to be used for location of a selected material and is otherwise referred to herein in the alternative as a “known” region, “reaction” region, “selected” region, “individual” region, or simply a “region.” The region may have any convenient shape, e.g., circular, rectangular, elliptical, wedge-shaped, etc. A discrete region and, therefore in some embodiments, the area upon which each distinct material is synthesized is smaller than about 25 cm², preferably less than 10 cm², more preferably less than 5 cm², even more preferably less than 1 cm², still more preferably less than 1 mm², and even more preferably less than 0.5 mm². In most preferred embodiments, the regions have an area less than about 10,000 μm², preferably less than 1,000 μm², more preferably less than 100 μm², and even more preferably less than 10 μm². In general, the regions are spatially addressable. In certain embodiments, the regions are discrete. For instance, the regions are separated from each other so that a material in a first region cannot interdiffuse with a material in a second region and thus the regions have a minimum size. This separation can be accomplished in many ways, which are discussed below. In other embodiments, the regions are continuous.

Porous Substrate: A substrate that is fluid permeable over at least a portion of its volume and which is further defined by a porous material having a support surface such that fluids can flow into or through the material. In many embodiments, at least one surface of such substrate will be substantially flat (and the substrate will contain no discrete regions), although in some embodiments it may be desirable to physically separate regions for different materials with, for example, dimples, wells, raised regions, etched trenches, or the like. In some embodiments, the substrate itself contains wells, raised regions, etched trenches, etc., which form all or part of the regions (for example a microtiter plate). The regions may be coated or otherwise treated over some or all of its surfaces or not. The substrate may be rigid, semi-rigid or non-rigid (e.g., will not support its own weight). By way of example, the substrate may be a sheet or wafer, e.g., an elongated thin member, or it may be a member having a larger thickness (such as a plate with apertures defined therein or a tray containing an array of reaction sites or micro-reactors). The substrate may also be a substrate treated mechanically, magnetically, electrically, chemically or otherwise to define particular regions. For instance, a porous foam may be selectively compressed to define discrete regions of high and low densities, or treated to obtain differing hydrophobicities (e.g., for aqueous fluids) or other surface property differences. A preferred porous substrate is a porous substrate filter, and more particularly a filter sheet such as a filter fabric or filter paper. Moreover, it is possible that such a filter sheet may be sandwiched between two opposing plates (e.g., as part of a holder, a fluid collector, or both), the latter having apertures defined therein corresponding to regions. A porous substrate may also encompass the employment of a supported separation medium (e.g., glass beads disposed on or between a porous layer such as a frit). Porosity, surface texture or topology of the substrate may be varied as desired to provide a suitable amount of surface area and desired porosity for separation of solids from fluids. By “porous substrate”, also contemplated is a plurality of porous substrates that are arranged in spaced or contacting opposed layering relation to each other, or a plurality of porous substrates assembled in a generally common plane or layer.

In one aspect, this invention provides a method for screening a plurality of solid samples, including the steps of providing a fluidic medium for delivering a solid material sample to a porous substrate; separating the solid sample from the liquid medium by filtering with the porous substrate for depositing the solid sample on the porous substrate; and performing analysis of the solid sample while the sample resides on the porous substrate, and preferably without removing the solid sample from the porous substrate before analysis.

It will be appreciated that this invention lends itself well to providing useful methods for a combinatorial materials science research program for the discovery or characterization of solid phase materials. In this regard, the present invention can be employed to investigate any of a number of different classes of sample materials including but not limited to electronic materials, optical, thermoelectrics, semiconductors, conductors, dielectrics, superconductors, magnetics, supermagnetics, piezoelectrics, battery electrodes, phosphors, fuel cell materials, pharmaceuticals, pharmaceutical polymorphs, high strength materials or other classes of metals, ceramics, composites, or polymeric materials. The system may also be used to analyze organic materials generally, for example, DNA, proteins, amino acid polymers, polysaccharides, nucleic acid polymers, salts of small organic molecules or other non-biological or biological materials.

In another embodiment, the sample materials are to be examined for use as potential catalysts, and thus they will typically include a catalyst precursor and at least one inorganic compound that is chemically inert or catalytic, preferably one containing a metal (e.g., an oxide, nitride, carbide, sulfate, phosphate) or active carbon, and still more preferably a ceramic. In a highly preferred embodiment, at least one ingredient is a metallic salt, or oxide, such as a known catalyst carrier or support.

In one aspect of the present invention, the material samples will be generally inorganic, and more preferably will include at least two ingredients, namely, at least a first ingredient and a second ingredient. It will be appreciated by the skilled artisan that the ingredients may be selected so that the resulting material will typically have a metal or metalloid element selected from the group consisting of Groups 1-17, Lanthanides and Actinides of the Periodic Table of Elements. More specifically, the resulting material preferably will include one or more element selected from the group consisting of Li, Na, K, Rb, Cs, Mg, Ca, Sr, Ba, Sc, Y, La, Ti, Zr, Hf, V, Ta, Cr, Mo, W, Ru, Os, Ir, Fe, Ni, Pt, Co, Cu, Ag, Au, Zn, Cd, Rh, Pd, P, As, S, Se, Te, Mn, Nb, Re, B, Al, Si, Ga, Ge, In, Sn, Sb, Tl, Pb, Bi, Lu, Ce, Pr, Nd, Sm, Eu, Gd, Tb, Dy, Ho, Er, Tm, Yb, Be, Hg, Pm, B, C, N, and mixtures thereof. Compounds of the forenoted elements such as (for example) oxides, nitrides, borides, carbides, sulfates, phosphates or mixtures thereof are also contemplated. Unless otherwise specified, for purposes of the present discussion, the use of a metal (e.g., in a fluid medium) also encompasses the use of the ionic form (e.g., salt) of the metal.

In another preferred embodiment, the material samples of the present invention are pharmaceutical polymorph compounds (which may also include solvation or hydration products, e.g., pseudopolymorphs), and the invention is employed in some or all aspects of solid-state polymorphism, including crystallization, phase transformations, and crystal structure resolution.

In this regard, as with other applications discussed herein, it is contemplated that the conditions under which the methods of the present invention are employed may be varied in an effort to replicate temperature, time, pressure or other conditions to which the sample materials may encounter in a commercial or industrial environment. Further, in connection with the research methodologies of the present invention, the present invention also contemplates that single crystals may be grown and analyzed.

In general, though one aspect of the present invention contemplates rapid synthesis and characterization of individual samples in isolation, the method and system of the present invention preferably contemplates forming a library of a plurality of the same or different materials using rapid-serial synthesis techniques, parallel synthesis techniques or a combination thereof. Specifically, individual material samples or plural member libraries of material samples can be prepared as described herein, and subsequently screened while resident on the porous substrate, preferably without having to further transfer off of the substrate or otherwise handle or disturb any of the individual materials.

In the formation of libraries in accordance with the present invention, one or a plurality of ingredients may be selected to form a desired material or may be selected to explore a compositional range or phase space potentially useful as a desired material. Ingredients are typically selected from commercially available atoms, molecules, compounds or complexes having a desired element. Ingredients typically are in a solid or liquid state, but may also be provided in a gaseous state.

Though examples are provided in the preceding discussion, selection of the ingredients will depend largely upon the intended use of the ingredient. It is preferred that one or both of the ingredients are provided in a flowable medium (e.g., as a liquid, solution, suspension, dispersion, emulsion, sol, gel, etc.) for deposition onto a porous substrate. Filtration is performed by the porous substrate. The first ingredient and second ingredient, optionally following further processing and/or treatment, can be subjected to reactive conditions, in the presence of reactant materials. Their properties can then be analyzed.

In one preferred embodiment, the solid phase material samples of the present invention may be provided in a liquid medium for deposition onto and filtration by a porous substrate, preferably by a filter sheet Any suitable liquid medium may be employed. The liquid medium may be tuned as desired with appropriate agents to alter its viscosity, surface or wetting characteristics to facilitate deposition of such medium onto the substrate. In one highly preferred embodiment, the materials of a library are precipitates obtained from mixing of ingredients in liquids. An example of one such preferred system is disclosed in U.S. Patent Application Entitled “A Method For Synthesizing Arrays of Materials”, Ser. No. 09/633,255 (Filed Aug. 7, 2000), hereby incorporated by reference. Thus, for example, at least two liquids are admixed optionally in the presence of a precipitating agent, and using a stirring member or the force derived from introduction of the liquids themselves. Solid precipitates form during the co-precipitation and can be separated from the liquid.

Particle sizes of any suspended particulates or resulting precipitates may range from as low as about 0.001 μm to about 300 μm, more preferably from about 0.1 μm to about 100 μm, and most preferably from about 1 μm to about 20 μm.

The liquids for delivering the solids may by provided immediately upstream from the porous substrate used for separating liquids from solids. Dispensing, mixing or other preparation of the sample materials may occur in the same vessel as the porous substrate. However, dispensing, mixing or other preparation may occur in a separate vessel with transfer of material to the vessel with the porous substrate.

It should be appreciated that the above is not intended as limiting and the present invention also is suitable for characterization of materials that are recrystallized from their liquid state, such as by the cooling or evaporation of solutions, or the inclusion of an anti-solvent, to result in a solid dispersed in a liquid supernatant. Further, the skilled artisan will appreciate that the present illustrations are not intended as restrictive, and that many variations of the substrate structures, providing steps and sequences of providing, are possible, all within the scope of the present invention. For example, the order in which the first and second ingredients are introduced may be varied. Elements or compounds of the first or second ingredients may be introduced into the other ingredient prior to introduction of the ingredient to the substrate. The first ingredient may be impregnated into the second ingredient, or vice versa.

As will be appreciated, samples prepared or processed in accordance with the above can, optionally, be further processed and/or treated (before, during or after deposition on the porous substrate, or even after characterization by beam radiation) as necessary through one or more steps (e.g., reaction, drying, calcining, sintering or otherwise heat-treating). For example, after separating in accordance with the invention discussed herein, the solid may be separated from the supernatant liquid and washed as needed, such as with a solvent. Any such wash or solvent may be removed and the resulting wet solid then dried in a suitable manner, such as evaporation by air drying, drying under a stream of inert gas, drying in a vacuum, or drying by heating. Alternatively, or in addition, an absorbent material, such as a wicking material, may be placed in contact with the sample for removal of liquids.

Other treatments may involve separate treatment of each of the components individually or together as a mixture. Other variations are also possible. For instance, in one embodiment, the library materials are treated to have the ability to adhere to regions on the substrate (whether coated, physically divided into regions or not). For instance, a suitable amount of a binder, adhesive or like agent might be added to assist in adhesion. In this manner, after characterizing the structure of the materials in the library according to the present invention, it may be possible further handle and use the materials for other screens or in connection with a further chemical reaction.

In addition, prior to screening (or even in the earlier deposition steps) the amount of material or its density in a region of a substrate can be varied as desired, through one or more steps of measuring, adding, packing, or physically removing materials (e.g., grinding or scraping) according to predetermined parameters.

In creating libraries in accordance with the present invention, it is frequently desirable to vary the compositions or stoichiometry of the starting materials. It is also possible to vary the reaction environment conditions from region to region to create different materials or materials with different properties. By way of illustration, with particular reference to the selection of the chemistry of a first and second different ingredient, it is possible that the first ingredient is constant across the substrate, but the second ingredient is varied region to region. Likewise it is possible to vary the first ingredient across the substrate, but maintain the second ingredient constant. Moreover, it is possible to vary both the first and second ingredients across the substrate.

Examples of ratios and techniques for forming a variety of libraries are illustrated in U.S. patent application Ser. Nos. 09/156,857 and 09/156,827 entitled “Formation of Combinatorial Arrays of Materials Using Solution-Based Methodologies,” hereby incorporated by reference. Preferably a library is created having at least 4 different materials, more preferably at least 5, still more preferably at least 10. Amounts of different materials in excess of 10 are contemplated for a single library in accordance with the present invention. For instance, libraries may contain at least 12, 24, 36, 48, 96, 256, 500, 1000, 10⁵, or 10⁶ different materials. In some embodiments, the library can include 96×N different materials, where N ranges from 1 to about 20, and preferably from 1 to about 10 or from 1 to about 5.

By way of illustration, if there is a two-ingredient material being prepared, a phase space is formed to examine the complete range of ingredient variation. A first library may be formed by selecting an amount consistent with the size of the region being used (discussed below) and mixing an appropriate molar amount of ingredient A and ingredient B so that the first region of the substrate contains 100% of ingredient A and 0% of ingredient B. The second region may contain 90% of ingredient A and 10% of ingredient B. The third region may contain 80% of ingredient A and 20% of ingredient B. This is repeated until a final region contains 0% of ingredient A and 100% of ingredient B. Library formation in this fashion applies to as many ingredients as desired, including 3 ingredient materials, 4 ingredient materials, 5 ingredient materials, 6 or more ingredient materials, or even 10 or more ingredient materials. Like techniques may be employed in preparing libraries having stoichiometry, thickness or other chemical or physical gradients.

Moreover, in another embodiment of the present invention, a method is provided for forming at least two different libraries of materials by delivering substantially the same ingredients at substantially identical concentrations to regions on both first and second substrates and, thereafter, subjecting the ingredients on the first substrate to a first set of reaction conditions or post-delivery processing or treating conditions and the ingredients on the second substrate to a second set of reaction conditions or post-delivery processing or treating conditions. Using this method, the effects of the various reaction parameters can be studied and, in turn, optimized. Reaction, processing and/or treatment parameters which can be varied include, for example, solvents, temperatures, times, pressures, the atmospheres in which the reactions, processing or treatments are conducted, the rates at which the reactions are quenched, etc. Other reaction or treatment parameters that can be varied will be apparent to those of skill in the art. Hence, one embodiment of the invention is where a library of materials, after its formation, is thereafter subjected to further processing (such as heat treating in an alternative atmosphere) to create an library of different materials.

The library can have as many materials as there are regions on the substrate. For purposes of this invention, the number of materials is typically equal to the number of regions on the substrate, unless certain regions are left empty. The number of regions on the substrate is discussed below, but applies as well to the number of materials.

In some embodiments, a region on the porous substrate is smaller than about 25 cm², preferably less than 10 cm², more preferably less than 5 cm², even more preferably 2 cm², still more preferably less than 1 cm², and still more preferably less than 0.5 cm². In most preferred embodiments, the regions have an area less than about 10,000 μm², preferably less than 1,000 μm², more preferably less than 100 μm², and even more preferably less than 10 μm². In this manner, it is possible that relatively small sample sizes can be employed, such as on the order of about 100 micrograms to about 500 mg, more preferably about 5 to about 50 mg.

Delivery of the material to a porous substrate in accordance with the present invention can be accomplished with any of a number of manual or automated methods. One preferred method and system for generating a combinatorial library and performing materials research with the library involves the employment of automated systems driven by suitable software, such as LIBRARY STUDIO™, by Symyx Technologies, Inc. (Santa Clara, Calif.); IMPRESSIONIST™, by Symyx Technologies, Inc. (Santa Clara, Calif.); or a combination thereof. The skilled artisan will appreciate that these systems can be adapted for use in the present invention, taking into account the disclosures set forth in commonly-owned copending U.S. patent application Ser. Nos. 09/174,856 and 09/305,830, each of which is hereby incorporated by reference.

Prior to delivering ingredients, mixing may be desired in preparing samples or libraries. Mixing is accomplished in any one of many manual or automatic methods. Mixing can be manual such as by shaking the vessel or well. Mixing can also be automatic such as by using an inert ball bearing in a shaken vessel or array of vessels, such as a titer plate. Mixing can also be accomplished via a dispenser that repeatedly aspirates and dispenses some or all of the contents of a vessel or well. In a preferred embodiment, mixing is performed in the nozzle of an automatic dispensing robot that repeatedly aspirates and dispenses some or all of the contents of a vessel or well. Other mixing methods include agitation of the solution with a gas stream, diffusion, sonication or other agitation techniques known to those skilled in the art.

By way of illustration, without limitation, a system for preparing a sample or library of samples in accordance with the present invention, includes a container for liquid to be dispensed, a pump system in pumping communication with a valve system. The valve system includes one or more valves (e.g., solenoid valves, such as Microdrop Model 3000 available from BioDot Inc.) adapted so that liquid from the container can be drawn into a dispenser (e.g., a syringe or ink jet dispenser having a nozzle) connected to the valves from negative pressure generated by the pump system. The liquid in the container can then be dispensed onto a substrate, which is preferably held on a mounting surface of a motion plate. In one preferred embodiment, the valve system portion including dispensers is movable in the x, y and z directions and the mounting surface and motion plate is movable in at least the x and y directions, thereby permitting degrees of freedom in the design and creation of spatially addressable samples in an array. The LIBRARY STUDIO™ brand software allows for interface with the pumping system to control dispensing amounts, according to predefined amounts. The IMPRESSIONIST™ brand software in turn controls the translation of the motion plate so that desired compositions or gradients can be prepared at predetermined locations on the substrate.

In some embodiments, the delivery process is repeated to provide materials with as few as two ingredients, although the process may be readily adapted to form materials having 3, 4, 5, 6, or even 10 or more ingredients therein. The density of regions per unit area will be greater than 0.04 regions/cm², more preferably greater than 0.1 regions/cm², even more preferably greater than 1 region/cm², even more preferably greater than 10 regions/cm², and still more preferably greater than 100 regions/cm². In most preferred embodiments, the density of regions per unit area will be greater than 1,000 regions/cm², more preferably 10,000 regions/cm², and even more preferably greater than 100,000 regions/cm².

Using the dispenser systems discussed in commonly owned U.S. patent application Ser. No. 08/327,513, incorporated by reference, the individual ingredients or component mixtures can be delivered separately to regions on the substrate either sequentially or simultaneously. In a presently preferred embodiment, the ingredients or component mixtures are sequentially delivered to either a single predefined region on the substrate or, alternatively, to multiple predefined regions on the substrate. For example, using a dispenser having two nozzles, one or more first ingredients can be delivered to regions on the substrate. Alternatively, using this same dispenser, an ingredient can be simultaneously delivered to two different regions on the substrate. In this instance, the same ingredient or, alternatively, two different ingredients can be delivered. If the same ingredient is delivered to both of the regions, it can be delivered at either the same or different concentrations. Similarly, using a dispenser having eight or more nozzles, for example, eight or more different ingredients can be simultaneously delivered to a single region on the substrate or, alternatively, eight or more ingredients (either the same or different) can be simultaneously delivered to eight or more different regions on the substrate.

Other systems may be employed as desired, including automated fluid dispensing systems. For example, the use of a fully automated fluid dispensing system is preferred for use in depositing the samples of the present invention, which typically will be provided in a liquid medium. Examples of suitable commercially available automated liquid dispensing systems include those offered by CAVRO Scientific Instruments (e.g., Model NO. RSP9652) or BioDot (Microdrop Model 3000). The fluids delivered by any dispensing technique will be introduced through passageways of the sample collector described herein. Thereafter, a suitable (positive or negative) pressure may be applied to flow the fluid through a porous substrate (e.g., a filter sheet), where solids will be captured by filtration and retained for later treatment, testing or both.

It should also be recognized from the above that the methods and apparatus of the present inventions are directed to separations achieved primarily by filtration. In this regard, it should be kept in mind that nothing herein precludes the employment of the present invention in combination with other separation techniques, such as chromatographic separation, sorbent trapping, or combinations thereof. In chromatography, there is typically a sample-containing mobile phase applied across a stationary phase, resulting in the time-resolved and spatially distributed separation of components of the sample, with ultimately the components being temporally and spatially distributed in the mobile phase for detection (typically in a flow detector). In sorbent “trapping”, selective isolation occurs of one or more components that make up some subset of the total components of the sample, without an independent mobile phase that is used for both sample contact with the sorbent, and sample elution off of the sorbent. The present invention, nonetheless, is able to achieve separations by filtration, that is based mainly upon physical or size separation, with generally incidental (if any) chemical interaction such as absorption, adsorption, or otherwise. Further, the present invention also achieves separation in the absence of a continuously applied mobile phase. In some embodiments, there can be an essential absence of such chemical interactions such that separation is effected primarily or completely by physical or size separation.

As discussed herein, in one highly preferred embodiment, the materials are characterized by reference to the existence of or the identity of their respective crystallographic structures. Rapid throughput is possible and the system lends itself well to employment with a variety of screening techniques.

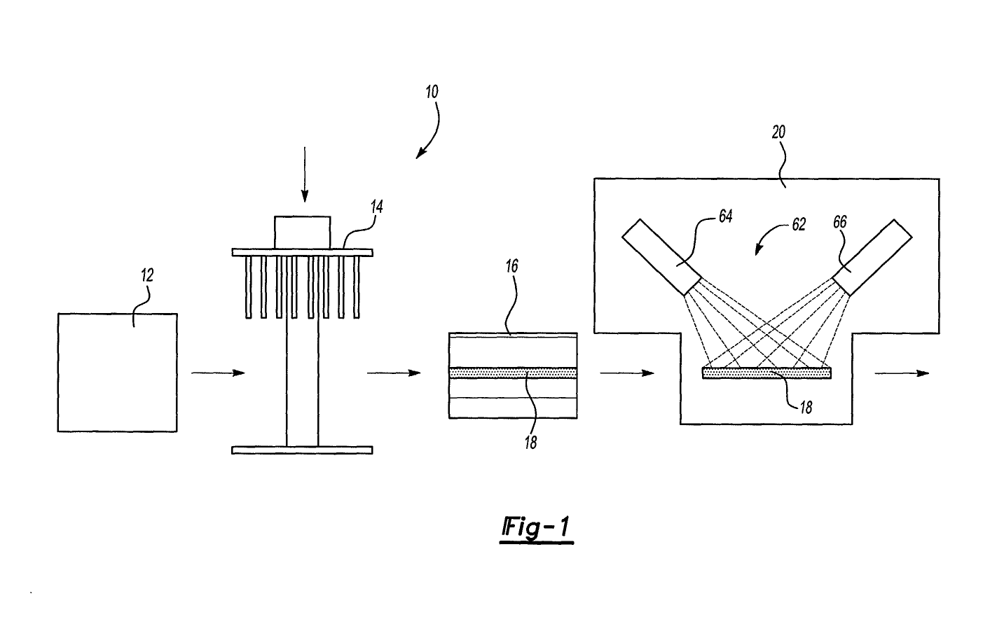

Referring to FIG. 1, there is shown an overview of one preferred research system 10 in accordance with the present invention. In a preferred embodiment, the system 10 includes one or more synthesis apparatus 12 for combinatorially producing fluidic materials containing solids or solid precursors. Such solids or solid precursors, upon separation from the fluidic medium, become samples or members of a combinatorial library. A suitable dispensing apparatus 14 may be employed for transferring the sample materials from the synthesis apparatus 12 to a sample collector 16. The sample collector 16 includes a porous substrate 18, into which the fluid materials are flowed, and onto which the samples are deposited or onto which a combinatorial library is formed. While the samples are on the porous substrate, and without the need to transfer them to a different substrate, they can be examined with a suitable x-ray analytical device 20 or with another suitable instrument.

Thus, one process of the present invention contemplates synthesizing fluid materials having a solid or solid precursor therein. The fluid materials are dispensed into the sample collector 16, and a solid sample is separated from its fluidic medium. Individual samples or a library of samples can be prepared on the porous substrate 18, which then may be analyzed while remaining on the substrate 18. For preparing libraries of sample members, it will be appreciated that the delivery and separation steps may be performed consecutively, simultaneously, or a combination thereof for the individual library members.

It will be appreciated that the practice of the present invention need not include each of the above components or steps. Components or steps can be combined or omitted as desired. Further, it is possible that the system may be an integrated assembly of some or all of the components, or the components may be configured as discrete stand-alone components.

The preparation and dispensing of samples having been discussed in detail in the above, and otherwise employing art-disclosed techniques, the discussion now turns to the particular sample collector and preferred analytical instrument.

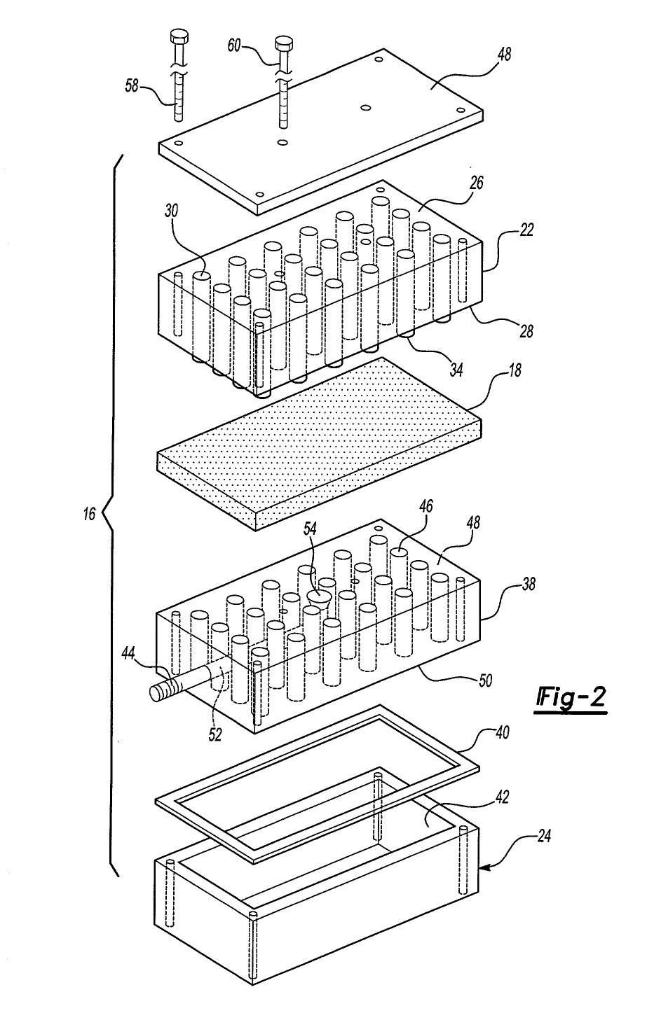

Referring to FIG. 2, an illustrative sample collector is shown as including a deposition guide block 22 for directing sample-containing fluids to the porous substrate 18, and a fluid receptacle 24, for capturing fluids separated from the sample by the porous substrate 18.

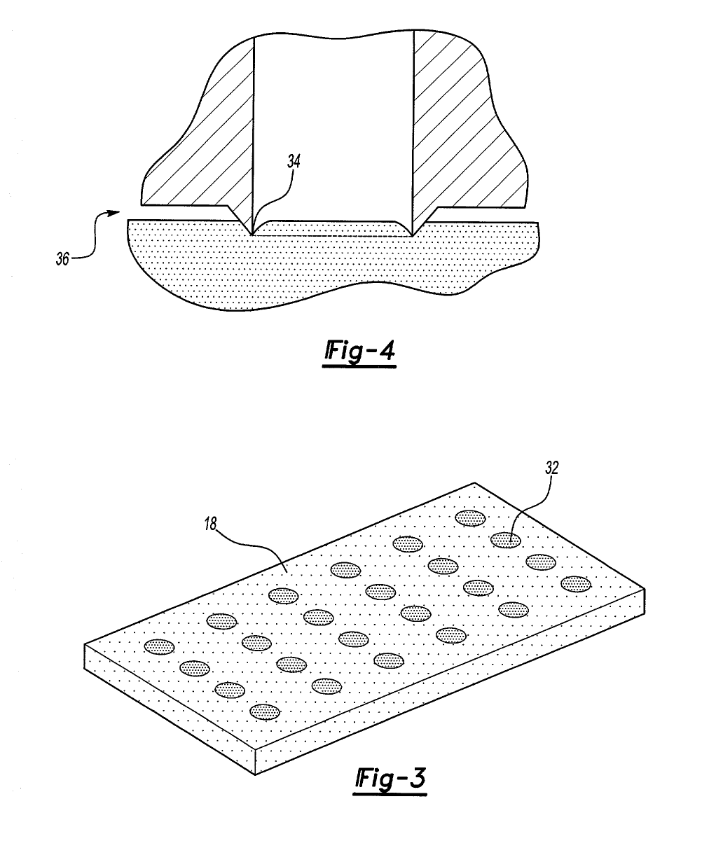

The deposition guide block 22 preferably is employed during deposition of a liquid sample onto the porous substrate 18. Preferably the guide block 22 has a first side 26 and an opposing second side 28, and includes a plurality of first suitable passageways 30 that guidingly permit the deposition of fluidic sample, so that discrete regions 32 (as depicted in FIG. 3) for solid sample collection can be achieved on the porous substrate 18.

As seen in both FIGS. 2 and 4, preferably the second side 28 is configured over its surface and about the peripheries of the respective first passageways 30 to have a knife-edge 34 for forming a substantially fluid tight seal 36 with the porous substrate 18. The taper of the knife-edge 34 may be adapted as desired depending upon the materials used, expected reaction conditions, reagents to employ, or other like factors. As illustrated in FIG. 4, the knife-edge 34 has approximately a 45° taper relative to the second side 28 of the block 22. Corresponding opposing or mating tapers may also be employed for the support plate 38. Of course, other seals may be used such as o-rings, gaskets or the like.

The porous substrate 18 is attached to one or more components, such as the deposition guide block 22, the fluid receptacle 24 or an optional support plate 38. One or more gaskets 40 (of any suitable material such as an elastomeric material (e.g., SANTOPRENE™), or the like) may be employed as desired to help seal the assembly. Of course, other intermediate holder structures may be employed as well.

The fluid receptacle 24 has an upstanding wall structure 42. The support plate 38, the receptacle 24, or both may have a suitable port configuration through which a negative pressure can be applied or through which fluids may otherwise be drained. By way of example, in the embodiment of FIG. 2, a port is defined in a vacuum line fitting 44, which may be connected to a suitable vacuum pump (not shown). Such port is shown in the support plate 38, which may also have a plurality of second passageways 46 in generally registered alignment with the first passageways. However, a port might be located in the receptacle. Further there may be no passageways in the support plate, the support plate might be omitted, or the receptacle omitted (with drainage to another fluid collector). In another embodiment, optionally a port is defined in a drain structure, which is capable of receiving fluids obtained by gravity or another suitable force. For embodiments in which a negative pressure such as a vacuum is employed, the guide block 22 is provided with a cover 48 for sealing.

As shown in FIG. 2, the support plate 38 has a first side 48 and a second side 50. A tunnel 52 is defined in the plate 38, which extends from one or more tunnel end 54 defined in the first side 48, to the vacuum line fitting 44. In this manner a vacuum can be drawn immediately adjacent the porous substrate 18. Of course, other configurations are possible as well for drawing the vacuum. There may be a plurality of tunnels extending to one or a plurality of vacuum lines. Further, one or more tunnels may extend from the second side 50. A suitable gasket, or like member, may be employed or integrated to assist in sealing as well.

The fluid receptacle 24, the support plate 38 or the deposition guide block 22 may include lips, tangs, clips, clamps or some other like structure to which the porous substrate or one of the other components can be attached for filtration and crystallographic analysis. In a particularly preferred embodiment, the porous substrate is sandwiched between the sample deposition guide block 22 and the support plate 38 during sample deposition. The receptacle, support plate and guide block are held together with a suitable clamp, fastener, adhesive, or other like mechanical or chemical attachment.

The fluid receptacle 24, the support plate 38 or the guide block 22 may be made of any suitable material. Preferably a material that is substantially inert or otherwise non-reactive relative to the samples is employed, such as suitable metal, ceramic, or plastic. Further, optionally the material is hydrophobic. Examples of preferred plastics include polyester, PTFE (e.g., Teflon®), acetal, polypropylene, polysulfone, or the like. An example of another commercially available material is that available under the designation of HYDEX.™ Of course, any of the materials for the structures of the apparatus herein may be coated, laminated or other wise treated to locally modify surface properties.

It will be appreciated that the delivery of samples to a predefined region may alternatively employ the use of spraying systems to help direct or confine the components to a particular location on the porous substrate. Thus, suitable masking systems may be employed where desired in addition to or alternatively to the depostion guide block. See, e.g., U.S. Pat. Nos. 5,985,356 and 6,045,671, hereby incorporated by reference.

The porous substrate 18 as described herein preferably is a relatively thin filtration media, and may be one or more sheets of paper, a foam, woven fabric, unwoven fabric, compacted powder, rubberized cloth or the like. However, the substrate may be relatively thick as well (such as might occur, for example, if plural layers are used, if supported beads are used, or the like). Thus, while thicknesses on the order of about 0.01 to about 50 mm, more preferably about 0.1 to about 10 mm, and still more preferably about 0.1 to about 2.5 mm are possible, thicknesses of greater than 1 cm or less than 0.1 mm are also possible. Though the force of gravity alone may suffice and be employed, for applications employing a vacuum or a like pressure gradient source for drawing fluid into the substrate, preferably the substrate is made of a generally hydrophobic material such as PTFE for controlling or impeding liquid flow until a pressure gradient is applied. Other suitable materials include, without limitation, rubberized cloth, binder-free glass fiber, polypropylene, nylon, (meth)acrylic (e.g., an acrylic copolymer), polycarbonate, cellulose, aluminum oxide, as well as other organic or inorganic materials. Examples of commercially available materials include, without limitation, those available under the designation VERSAPOR™, ANOPORE™ or the like. Preferably, the porous substrate is such that it will, consistently and reproducibly, substantially avoid undesired background in the resulting x-ray analysis data. A preferred pore size for the porous substrate ranges from about 0.001 micron to about 1 mm, more preferably about 0.1 to about 500 microns and still more preferably about 5 to about 50 microns. Larger and smaller pore sizes are also possible.

The entire assembly of the sample collector 16 or respective components thereof may be held together in any suitable manner. In one preferred embodiment, a plurality of perimeter fasteners 58 are employed in combination with interior fasteners 60. Preferably, the length of the fasteners is controlled for avoiding reactive contact with fluids that enter the collector.

It will be appreciated that the samples are typically delivered to the porous substrate in a liquid medium after synthesis. However, synthesis may occur after or during delivery. Further, the property screened for may be derived from a post-delivery treatment step.

The porous substrates of the present invention may be recycled for further use. Or they may be disposable after one or more uses. It will be appreciated from the above that the present invention advantageously allows the use to gain quick access to such substrates and facilitates subsequent removal or handling of samples deposited upon the substrates. Further, it is possible that the porous substrate or another sample collector component may be integrally formed with one or more other sample collector components, or even with the fluid dispenser itself.

Any suitable analytical device may be employed for characterizing the samples, and more preferably for analyzing the crystal structure of each of the samples while the samples remain on the porous substrate. Preferably a beam radiation device is employed wherein a beam is focused a target sample (e.g., a library member), a detector measures the response of the target sample to the radiation. For a rapid serial analysis, the beam is advanced to another sample and the process is repeated. Parallel analysis is also possible by the simultaneous employment of a plurality of beams and a detection system for detecting the response of a plurality of samples to its respective beam radiation. It may also be possible to obtain rapid data acquisition using techniques such as concurrent x-ray analysis, such as disclosed in M. Ohtani, Applied Physics Letters, Vol. 79, page 3594-3596 (2001), hereby incorporated by reference.

Preferably the beam radiation device is a suitable collimated beam or microbeam radiation device, including but not limited to a device selected from conventional x-ray diffraction devices, electron radiation devices, neutron radiation devices, synchrotron radiation devices, other suitable microbeam radiation devices. In a preferred embodiment, the device will be a diffraction instrument 62, and thus, will include (as in FIG. 1) an x-ray or other suitable collimated beam source 64 and a detector 66 for detecting the response of the sample to the beam (e.g., by measuring diffraction, transmission, absorption, reflection, emission, scatter, or the like, of the radiation). Optionally, suitable optics are employed for aid in aligning and monitoring one or more members of the library under analysis. Preferably the x-ray source is capable of being collimated to different sizes in any suitable manner (e.g., with a pinhole collimator, monocapillary or the like), or intensified (e.g., with the use of mirrors). Further, a suitable monochromator may be employed. Examples of suitable x-ray instrumentation are disclosed in commonly owned, U.S. application Ser. Nos. 09/680,154; 09/215,417; and 09/667,119, hereby incorporated by reference. Thus, in one preferred embodiment, the beam from the device (upon emission) may be as small as about 25 μm. For example, in one embodiment, the beam width is about 1 mm, and has a divergence on the order of less than about 3 mm.

The detector may employ any suitable detection technique. In one embodiment, it is a suitable area detector capable of gathering information bearing upon phase analysis, particle shape, particle size, percent crystallinity, or the like. An example of a preferred commercially available x-ray diffraction analytical device whose components are readily adaptable for use in the present invention is the D-8 from Bruker AXS.

It will be appreciated that the orientation of the x-ray beam relative to the detector may be such that both are focused at an angle relative to the specimen analyzed. Other orientations are possible as well, such as opposing face-to-face orientation, where the detector detects beams transmitted through the sample.

In one embodiment, the sample collection system, one or more components of the x-ray diffraction analytical device or a combination thereof may be coupled with a suitable drive mechanism for positioning members of the library relative to the path of the x-ray beam. For example, a suitable multi-axis stepper or servo-motor may be employed and controlled by a microprocessor or otherwise computer controlled. In this manner, rapid analysis of a library of samples can be accomplished by a succession of steps that include focusing a radiation beam on a target library member, optionally rotating the beam or a detector through an arc (or otherwise translating it) while maintaining focus on the target library member, detecting radiation diffracted by the library member throughout any range of rotational angles, and then advancing the beam to another library member and repeating the steps. This sequence is repeated until all members of the library have been analyzed. As mentioned, it is also possible to have opposing face-to-face relation between the beam and detector, with analysis performed for a sample and then translation of the library relative to the beam/detector position. Thus, wide angle or small angle scattering may be employed.

For each sample, the information obtained preferably is inputted and stored into a computer, which can retrieve such information for subsequent analysis or comparison with other library members.

Samples may be heated or otherwise treated before or during analysis. For example, the entire system, including the analytical device, may be enclosed in a single chamber, whose atmosphere (including temperature, pressure, or the like) is separately controllable. Alternatively, some or all of the analytical device may be disposed outside a suitable chamber having an transparent window for allowing measurements to be made from a point outside the chamber. In one embodiment, it is possible to aim a beam at a library member that is functioning as a supported catalyst in the presence of reactants and under reaction conditions. The crystallographic time response of the catalyst might be measured in this manner.

Data may be outputted in any suitable manner. For example, in the context of x-ray analysis, it may take the form of a conventional x-ray diffraction pattern having peaks corresponding to various angles (e.g., a diffractogram).

While the above has been discussed in the context of rapid serial analysis, it is not intended as so limiting parallel analysis may also be employed through the simultaneous or parallel use of plural analytical devices.

The libraries in accordance with the present invention lend themselves to the testing of diverse properties in addition to or alternative to crystallographic properties. Thus, in addition to or alternative to beam radiation analysis, such as x-ray ray diffraction, the libraries can be screened while on the porous substrate (or removed therefrom) using infrared techniques, thermal analysis techniques (such as differential scanning calorimetry, differential thermal analysis or the like), chromatographic techniques, resonance, spectroscopy, light scatter, spectrometry, microscopy, nuclear magnetic resonance, optical measurements, electrochemical measurements. By way of example, X-ray diffraction (XRD) and X-ray fluorescence (XRF) can be used in combination to determine the material crystal structure and composition, respectively. Other suitable screens might be gleaned from commonly owned U.S. Pat. Nos. 5,776,359; 5,959,297; 6,013,199; 6,034,775; 6,087,181; 6,151,123; 6,157,449; 6,175,409; 6,182,499; and 6,187,164 (all of which are hereby incorporated by reference), as well as other commonly owned patent properties. It is also possible that simultaneous screens may be employed while the libraries are on the porous substrate, such as (for instance) x-ray diffraction and one or more of the above-listed screens. Thus, it can be seen how those of skill in this art can effectively utilize the methods of this invention for a combinatorial materials science research program.

Another aspect of the present invention involves correlating the data received from the x-ray analysis or other screen with information known about ingredients of each of the materials, processing conditions of each of the materials or a combination thereof. The respective samples of one or more libraries can be compared with each other based upon the data and ranked. In this manner, a large field of research candidates can be narrowed to a smaller field by identifying the candidates that perform better than others with respect to a predetermined property, structure, or figure of merit. Comparative review of results might lead to rankings of performance from better to worse, or the like. Likewise, a large field of research candidates can be narrowed to a smaller one by identifying those that meet a certain predetermined criteria (e.g., whether a crystal structure is formed). Further libraries can be prepared for further analysis. Alternatively, bulk quantities of materials having the desired properties or structures can be made for commercial applications. Data analysis may be performed manually, or by semi-automated or automated techniques. For example, it is possible to employ either or both of the LIBRARY STUDIO™ (from Symyx Technologies,Inc.) and IMPRESSIONIST™(from Symyx Technologies,Inc.) for library design and synthesis, and POLYVIEW™(from Symyx Technologies,Inc.) or other suitable data management software to assist in correlating the data. Further, it is contemplated that data obtained from the use of the present invention can be used to develop data bases, such as a crystallography data base, or can be used for further interpretation or modeling.

One of the advantages of the present invention is that libraries can be provided with liquids, deposited on a porous substrate (e.g., a porous substrate) and tested, while still on the substrate, following their preparation (and without an additional transfer off of the substrate or other additional handling of the supported materials). The materials can also be screened, in alternative embodiments, with the materials within the same chamber where the library is synthesized, without the need to transfer to an external test site.

The present invention thus allows many materials (e.g., greater than 4) to be tested rapidly without the need to remove the substrate from the test apparatus, or replace it with a different substrate. In this manner, the achievement of large amounts of data is possible over a short period of time. For example sample preparation throughputs from initial preparation through deposition onto the porous substrate are possible of no longer than 10 minutes per sample, more preferably, no longer than 3 minutes per sample, and still more preferably no longer than 1 minute per sample. Further, libraries on a single substrate of at least 4 members can go from liquid medium to screening at a rate less than 40 minutes per library, and more preferably less than 10 minutes per library. Moreover, because the amounts needed for screening are relatively small, (e.g., less than 1 gram, and preferably less than 1 milligram), time and expense savings on sample preparation are also realizable. The length of time for performing the x-ray analysis preferably is less than 5 minutes per sample, and more preferably less than about 1 minute per sample. However, suitable results may be obtained with times on the order of about 1 second per sample, preferably on the order of about 10 seconds per sample.

As can be readily appreciated from the above, the present invention finds suitable application in any of a number of different sample preparation scenarios. For example, without limitation, the present invention can be employed for filtering precipitate from a liquid medium, (e.g., such as that which might result from the practice of U.S. Patent Application Entitled “A Method For Synthesizing Arrays of Materials”; Ser. No. 09/633,255; Filed Aug. 7, 2000, hereby incorporated by reference. Of course, it may be employed for filtration of suspended or dispersed particles (e.g., from a slurry or other suspension). It may be employed for separating materials in emulsions. Other applications will also be readily apparent to the person skilled in the art.

For preparing one or more of the above, the specific synthesis protocol may be varied. For example, an anionic solution may be provided, which upon contacting with a cationic solution, precipitates as a solid that can be filtered (e.g., by applying an overpressure, or alternatively a vacuum, to the apparatus of the present invention). Precipitation may also be achieved by cooling a liquid (e.g., a saturated solution, or one to which an anti-solvent has been added).

The reagents employed may be organic, inorganic, acid or basic. Any of a variety of solvents may be used, including for example, water, alcohols, ethers, ketones, aldehydes, aromatics, halogenated solvents (e.g., chlorinated), other polar solvents, or the like.

It will also be appreciated that some or all of the components of the apparatus of the present invention can be suitably employed in practice of other prefiltration or post-filtration steps. For example, in one embodiment, samples can be mixed, heated, cooled or otherwise processed while in the passageways 30, 46 or both. It is also possible (e.g., by flipping the apparatus over by 180°, introducing a barrier layer or otherwise) to collect liquids from the samples within the passageways into discrete vessels, rather than into the fluid receptacle 24.

It will also be appreciated from the above that the present invention is not limited to filtering of materials provided initially solely in a liquid state. For example, in one embodiment, a solid phase may be transferred to the apparatus of the present invention using solid handling robots or other suitable instruments, then dispersed (preferably evenly) on the porous substrate through the preparation of a suitable suspension, and followed thereafter by filtration.

From the above, it will be readily appreciated how the present invention advantageously is employed in the rapid analysis of one or a plurality of newly synthesized but uncharacterized materials. The invention may also be employed for the rapid characterization of existing known materials. In another embodiment, a combination of known and unknown materials are rapidly characterized, such as by the use of a reference control or standard in a library of materials. Although the invention has been described with particular reference to certain preferred embodiments thereof, variations and modifications can be effected within the spirit and scope of the following claims.

What is claimed is:

1) A method for X-ray characterization of a solid material sample, comprising the steps of:
1) providing a liquid medium for delivering a solid material sample to a porous substrate filter;
2) separating said solid sample from said liquid medium by filtering with said porous substrate so that said solid sample is deposited on said porous substrate filter;
3) characterizing said solid sample by performing x-ray analysis of said solid sample while said sample resides on said porous substrate filter and without removing said solid sample from said porous substrate filter before said x-ray analysis; and
4) repeating said steps (1)-(3) for analyzing a library of a plurality of solid material samples.

2) A method for characterization of a solid material sample, the method comprising;
1) simultaneously filtering at least four solid material samples in a fluidic medium through a porous substrate to separate said solid material samples from a fluidic medium;
2) depositing said at least four solid materials onto their own respective region on said porous substrate, and
3) characterizing said deposited solid materials by detection of beam radiation while said solid materials reside on said porous substrate and without removing said solid material samples from said porous substrate.

3) A method for characterization of a solid material sample, the method comprising:
1) providing an analytical system including:
a. a substrate holder having at least four apertures and being adapted to receive and support a porous substrate having a first side and a second side, said substrate holder being further adapted to provide for fluid communication between said samples and said first side of said porous substrate;
b. a receptacle having at least one collection cavity, said receptacle being adapted to provide fluid communication between said collection cavity and said second side of said porous substrate;
c. a source for providing a pressure gradient across said porous substrate, such that said samples in fluid communication with said first sides of said porous substrates can be simultaneously filtered through said porous substrates to separate solid-phase components of said samples from liquid phases thereof, and thereby deposit said solid phase components of said samples on said first side of said porous substrate; and
d. a characterization instrument adapted for analysis of said deposited solid-phase components while said solid-phase components reside on said porous substrate;
2) placing a porous substrate on said substrate holder;
3) simultaneously filtering at least four solid material samples through a porous substrate to separate said solid material the samples from a fluidic medium;
4) simultaneously depositing said at least four solid materials onto their own respective region on said porous substrate, and
5) beam radiation characterizing said deposited solid materials by analysis while said solid materials reside on said porous substrate and prior to removing said solid sample from porous substrate.

4) The method of claim 1, wherein each of said steps (1) and (2) is performed simultaneously for each of said samples.

5) The method of claim 1, further comprising the step of correlating data received from said x-ray analysis with information known about said ingredients of each of said materials, processing conditions of each of said material samples or a combination thereof.

6) The method according to claim 1, further comprising the steps of narrowing a first field of research candidates to a second field of research candidates that is smaller than said first field, by identifying those material samples that meet a certain predetermined criteria.

7) The method according to claim 1, further comprising preparing further libraries for further analysis based upon information obtained from said x-ray analysis.

8) The method according to claim 1, further comprising preparing bulk quantities of materials based upon information obtained from said x-ray analysis.

9) The method according to claim 1, wherein at least four different solid samples are screened as part of a library of materials.

10) The method of claim 2, wherein said characterizing step includes performing consecutive x-ray diffraction analysis of each said material sample while said material sample is on said filter and without removal of said material from said filter before said x-ray diffraction analysis.

11) The method of claim 10, further comprising the step of correlating data received from said x-ray diffraction analysis with information known about said ingredients of each of said material samples, processing conditions of each of said material samples or a combination thereof.

12) The method according to claim 2, further comprising the steps of narrowing a first field of research candidates to a second field of research candidates that is smaller than said first field, by identifying those material samples that meet a certain predetermined criteria.

13) The method according to claim 10, further comprising preparing further libraries for further analysis based upon information obtained from said x-ray analysis.

14) The method according to claim 10, further comprising preparing bulk quantities of materials based upon information obtained from said x-ray diffraction analysis.

15) The method according to claim 2, wherein at least two of said at least four solid materials is a different composition relative to said others.

16) The method according to claim 2, wherein said at least four solid materials have unknown crystallographic properties prior to said x-ray analysis.

17) The method according to claim 2, wherein a reference standard is included as at least one of said at least four solid materials.

18) The method according to claim 3, wherein said porous substrate is hydrophobic over at least a portion of said first side of said porous substrate.

19) The method according to claim 18, wherein said porous substrate has a thickness of about 0.1 to about 2.5 mm.

20) The method according to claim 3, further comprising forming a seal between said substrate holder and said porous substrate.

21) The method according to claim 1, 2 or 3, wherein said fluidic medium is a liquid medium.

22) The method according to claim 1, 2 or 3, wherein said characterizing step is performed consecutively for each sample.

23) The method according to claim 1, 2 or 3, wherein said characterizing step includes a step of x-ray diffraction.
